(19) United States
(12) Patent Application Publication
(10) Pub. No.: US 2025/0205469 A1
(43) Pub. Date: Jun. 26, 2025

(54) CIRCULATORY SUPPORT SYSTEM

(71) Applicant: Boston Scientific Scimed, Inc., Maple Grove, MN (US)

(72) Inventors: Dov Goldvasser, Framingham, MA (US); Darrin Dale Beekman, Osseo, MN (US); Serena Scott, Worcester, MA (US)

(73) Assignee: Boston Scientific Scimed, Inc., Maple Grove, MN (US)

(21) Appl. No.: 18/987,862

(22) Filed: Dec. 19, 2024

Related U.S. Application Data
(60) Provisional application No. 63/624,867, filed on Jan. 25, 2024.
(60) Provisional application No. 63/613,302, filed on Dec. 21, 2023.

(51) Int. Cl. A61M 60/13; A61M 60/17; A61M 60/216; A61M 60/816

(52) U.S. Cl. CPC A61M 60/13; A61M 60/17; A61M 60/216; A61M 60/816; A61M 2205/04; A61M 2205/3303; A61M 2205/3327; A61M 2205/3334; A61M 2205/3375; A61M 2205/50; A61M 2210/125

(57) ABSTRACT

Example medical devices and methods for using medical devices are disclosed. A cardiac pump system includes a catheter shaft coupled to a cardiac pump which includes an impeller housing, a cannula and an impeller. The cannula includes a distal end region and a proximal end region, wherein the distal end region of the cannula is configured to be positioned in a heart. The cardiac pump system includes a first ultrasound transducer coupled to the catheter shaft, wherein the first ultrasound transducer is configured to directly measure a velocity of blood flowing adjacent to the first ultrasound transducer. The cardiac pump system includes a second ultrasound transducer coupled to the catheter shaft, wherein the second ultrasound transducer is configured to transmit a first ultrasound signal toward a wall of a body vessel and receive a reflected portion of the first ultrasound signal reflected from the wall of the body vessel.

### CROSS-REFERENCE TO RELATED APPLICATIONS

This application claims the benefit of U.S. Provisional Patent Application Ser. No. 63/613,302, filed Dec. 21, 2023 and U.S. Provisional Patent Application Ser. No. 63/624,867, filed Jan. 25, 2024, both of which are incorporated herein by reference.

### TECHNICAL FIELD

The present disclosure relates to percutaneous circulatory support device systems. More specifically, the disclosure relates to percutaneous circulatory support devices that include one or more sensors for sensing and measuring therapeutic parameters.

### BACKGROUND

Percutaneous circulatory support devices such as blood pumps can provide transient cardiac support in patients whose heart function or cardiac output is compromised. Such devices may be delivered percutaneously from the femoral artery, retrograde through the descending aorta, over the aortic arch, through the ascending aorta across the aortic valve, and into the left ventricle. Some percutaneous circulatory support devices may include one or more sensors for directly measuring various therapeutic parameters such as blood velocity and vessel diameter. Further, the direct measurement of various therapeutic parameters (e.g., blood velocity, vessel diameter, etc.) may be utilized to indirectly determine the position of the circulatory support device within the blood vessel and/or other cardiac parameters (e.g., cardiac output, ventricular ejection fraction, etc.). Accordingly, there is an ongoing need to provide circulatory support device systems including one or more sensors designed to directly sense and measure therapeutic parameters during a cardiac procedure. Circulatory support device systems including one or more sensors designed to directly sense and measure therapeutic parameters during a cardiac procedure are disclosed herein.

### SUMMARY

This disclosure provides design, material, manufacturing method, and use alternatives for medical devices and/or systems. An example cardiac pump system includes a catheter shaft having a distal end region coupled to a cardiac pump. Further, the cardiac pump system includes an impeller housing, a cannula and an impeller. Further, the cannula includes a distal end region and a proximal end region, wherein the distal end region of the cannula is configured to be positioned in a left ventricle of a heart. Further, the cardiac pump system includes a first ultrasound transducer coupled to the catheter shaft, wherein the first ultrasound transducer is configured to directly measure a velocity of blood flowing adjacent to the first ultrasound transducer. Further, the cardiac pump system includes a second ultrasound transducer coupled to the catheter shaft, wherein the second ultrasound transducer is configured to transmit a first ultrasound signal toward a wall of a body vessel and receive a reflected portion of the first ultrasound signal reflected from the wall of the body vessel.

Alternatively or additionally to any of the embodiments above, further comprising a console coupled to the catheter shaft, wherein the console includes a processor, and wherein the console is configured to receive the reflected portion of the first ultrasound signal from the second ultrasound transducer.

Alternatively or additionally to any of the embodiments above, wherein the processor is configured to utilize the reflected portion of the first ultrasound signal to determine a diameter of the body vessel adjacent to the second ultrasound transducer.

Alternatively or additionally to any of the embodiments above, wherein the processor is configured to calculate a flow rate of blood passing through the body vessel based on the velocity of blood measured by the first ultrasound transducer and the diameter of the body vessel determined by the processor.

Alternatively or additionally to any of the embodiments above, wherein the first ultrasound transducer is positioned adjacent to the second ultrasound sensor.

Alternatively or additionally to any of the embodiments above, wherein the first ultrasound transducer and the second ultrasound transducer are attached to an outer surface of the catheter shaft.

Alternatively or additionally to any of the embodiments above, wherein the first ultrasound transducer and the second ultrasound transducer are attached to a housing attached to the catheter shaft.

Alternatively or additionally to any of the embodiments above, wherein the first ultrasound transducer, the second ultrasound transducer or both the first ultrasound transducer and the second ultrasound transducer extend at least partially into a wall of the catheter shaft.

Alternatively or additionally to any of the embodiments above, wherein the first ultrasound transducer is configured to utilize doppler ultrasound to directly measure the velocity of blood flowing adjacent to the first ultrasound transducer.

Alternatively or additionally to any of the embodiments above, wherein one or more optical fibers are utilized to directly measure the velocity of blood flowing adjacent to the first ultrasound transducer.

Alternatively or additionally to any of the embodiments above, wherein the second ultrasound sensor is configured to transmit a second ultrasound signal toward the body vessel wall and receive a reflected portion of the second ultrasound signal reflected from the vessel wall, and wherein the console is configured to receive the reflected portion of the second ultrasound signal from the second ultrasound transducer, and wherein the processor is configured to utilize the reflected portion of the first ultrasound signal and the reflected portion of the second ultrasound signal to determine a diameter of the body vessel adjacent the second ultrasound transducer.

Alternatively or additionally to any of the embodiments above, wherein the first ultrasound transducer includes a first transmission face configured to transmit a velocity ultrasound signal, and wherein the first transmission face tapers away from the catheter shaft such that the velocity ultrasound signal transmitted by the first transmission face propagates away from the catheter shaft at an angle relative to a longitudinal axis of the catheter shaft.

Alternatively or additionally to any of the embodiments above, wherein the first ultrasound transducer extends circumferentially around the catheter shaft.

Alternatively or additionally to any of the embodiments above, wherein the second ultrasound transducer includes an array of individual ultrasound transducer elements.

Another example cardiac pump system includes a catheter shaft having a distal end region coupled to a cardiac pump. The cardiac pump includes an impeller housing, a cannula and an impeller. Further, the cannula includes a distal end region and a proximal end region, wherein the distal end region of the cannula is configured to be positioned in a left ventricle of a heart. The cardiac pump system further includes a first ultrasound transducer coupled to the catheter shaft and a second ultrasound transducer coupled to the catheter shaft. Further, the first ultrasound transducer is positioned adjacent to the second ultrasound sensor. Further, the cardiac pump system includes a third ultrasound transducer positioned on an outer surface of the cannula.

Alternatively or additionally to any of the embodiments above, further comprising a fourth ultrasound transducer positioned on the outer surface of the cannula, and wherein the first ultrasound transducer and the second ultrasound sensor are coupled to a housing positioned on the catheter shaft.

Another example cardiac pump system includes a catheter shaft having a distal end region coupled to a cardiac pump. Further, the cardiac pump includes an impeller housing, a cannula and an impeller, wherein the cannula includes a distal end region and a proximal end region, wherein the distal end region of the cannula is configured to be positioned in a left ventricle of a heart. Further, the cardiac pump system includes a first ultrasound transducer coupled to the catheter shaft, wherein the first ultrasound transducer is configured to directly measure a velocity of blood flowing adjacent to the first ultrasound transducer. Further, the cardiac pump system includes a second ultrasound transducer coupled to the catheter shaft, wherein the second ultrasound transducer assembly is configured to transmit an ultrasound signal toward a wall of a body vessel and receive a reflected portion of the ultrasound signal reflected from the wall of the body vessel. Further, the cardiac pump system includes a third ultrasound transducer positioned on an outer surface of the cannula.

Alternatively or additionally to any of the embodiments above, wherein the third ultrasound transducer is configured to transmit an imaging ultrasound signal and receive a reflected portion of the imaging ultrasound signal reflected from blood or a body tissue.

Alternatively or additionally to any of the embodiments above, further comprising a fourth ultrasound transducer positioned on the outer surface of the cannula, and wherein the fourth ultrasound transducer is circumferentially spaced 180 degrees from the third ultrasound transducer.

Alternatively or additionally to any of the embodiments above, further comprising a console coupled to the catheter shaft, wherein the console includes a processor, wherein the processor is configured to generate a three-dimensional image based on the reflected portion of the imaging ultrasound signal from the third ultrasound transducer.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

FIG. 1 depicts a percutaneous circulatory support system, including a circulatory support device and its relative position in a heart of a patient;

FIG. 2 is a schematic block diagram of a console management system;

FIG. 3 depicts a portion of the circulatory support system including various transducers shown in FIG. 1 positioned in the heart of a patient;

FIG. 4 depicts an example velocity transducer coupled to a catheter shaft;

FIG. 5 depicts another example velocity transducer coupled to a catheter shaft;

FIG. 6 depicts another example velocity transducer coupled to a catheter shaft;

FIG. 7 depicts another example velocity transducer coupled to a catheter shaft;

FIG. 8 depicts an example ultrasound transducer coupled to a catheter shaft;

FIG. 9 depicts multiple ultrasound transducers coupled to a catheter shaft;

FIG. 10 depicts the ultrasound transducers of FIG. 9 positioned in a body vessel;

FIG. 11 depicts an ultrasound imaging transducer positioned along a portion of an example circulatory support device;

FIG. 12 is cross-section taken along line 12-12 of FIG. 11.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

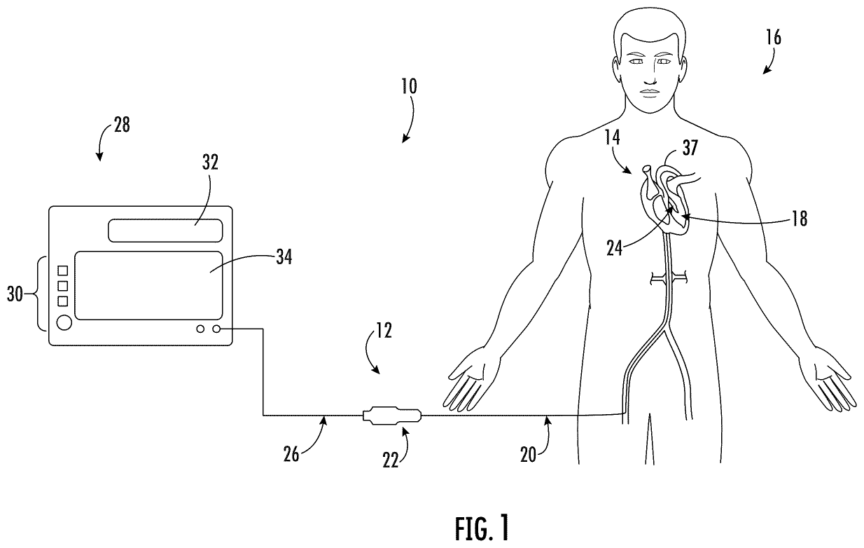

FIG. 1 illustrates an example percutaneous circulatory system 10 including a circulatory support device 12 positioned in the heart 14 of a patient 16. The circulatory support device 12 may include a flexible elongated catheter shaft 20 having a first end attached to a handle 22 and a second end attached to a blood pump 24. FIG. 1 illustrates the blood pump 24 positioned in the left ventricle 18 of the patient 16. The blood pump 24 may be delivered (e.g., tracked) to the ventricle 18 percutaneously over a guidewire. For example, the catheter shaft 20 and blood pump 24 may be tracked over a guidewire through the femoral artery, past the renal arteries and the descending aorta, over the aortic arch, through the ascending aorta 37, past the aortic valve and into the left ventricle 18.

FIG. 1 further illustrates that the handle 22 may include a distal end region attached to the catheter shaft 20 and a proximal end region attached to an electrical power cable 26. The electrical power cable 26 may include an end region connected to a console 28. It can be appreciated that the handle 22 may include one or more actuators (e.g., buttons, levers, dials, switches, etc.) designed to permit a clinician to control various functions of the blood pump 24. For example, a clinician may be able to control the speed of the motor and/or an impeller located in the blood pump 24 via actuation of one or more actuators located on the handle 22.

Additionally, FIG. 1 illustrates that the console 28 may include one or more control knobs (e.g., buttons, knobs, dials, etc.) 30 and/or one or more displays. For example, FIG. 1 illustrates the console 28 may include a first display 32 and a second display 34. It can be appreciated that the console 28 may include more than two displays. Additionally, while FIG. 1 illustrates the first display 32 and the second display 34 integrated into the console 28, it is contemplated that the circulatory system 10 may be designed such that the first display 32, the second display 34 or both the first display 32 and the second display 34 are separate, distinct components of the circulatory system 10. In other words, the first display 32, the second display 34 or both the first display 32 and the second display 34 may be separate stand-alone displays, apart from the console 28. In some examples, the first display 32 and the second display 34 may display data received from separate sources.

In some examples, the second display 34 may be designed to attach to the console 28 and/or the first display 32. For example, the first display 32 may be integrated into the console 28 while the second display 34 may be configured to attach to a portion of the console 28. In yet other examples, both the first display 32 and the second display 34 may be a separate stand-alone display whereby the second display 34 may be configured to attach to the first display 32, or wherein the first display 32 may be configured to attach to the second display 34.

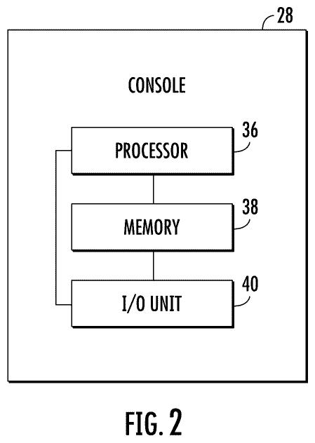

FIG. 2 illustrates that the console 28 may include, among other suitable components, one or more processors 36, memory 38, and an I/O unit 40. The processor 36 of the console 28 may include a single processor or more than one processor (e.g., a first processor 36 providing data/instructions to the first display 32 and a second processor 36 providing data/instructions to a second display 34) working individually or with one another. The processor 36 may be configured to execute instructions, including instructions that may be loaded into the memory 38 and/or other suitable memory. Example processor components may include, but are not limited to, microprocessors, microcontrollers, multi-core processors, graphical processing units, digital signal processors, application specific integrated circuits (ASICs), field programmable gate arrays (FPGAs), discrete circuitry, and/or other suitable types of data processing devices. In some examples, the processor 36 of the console may be configured to execute program instructions. Program instructions may include, for example, firmware, microcode or application code that is executed by the processor 36, a microprocessor and/or microcontroller. The one or more processors 36 may be configured to each manage different functions. They may also be configured to concurrently perform the same functions (e.g., redundant system). Further yet, they may be configured such that a first processor 36 performs a given function and a second processor 36 reviews the result of the function of the first processor 36 for correctness (e.g., command-monitor system) including accuracy and completeness.

In some examples, the first display 32 may be controlled primarily by the console's firmware control instructions and, therefore, may require relatively little processing power, relatively few instructions and very simple communication between the processor 36 and the display 32, compared to the second display 34 (e.g., a touch screen display 34), which may be controlled primarily by an embedded computer with a flexible and relatively complex communication protocol.

The memory 38 of the console 28 may include a single memory component or more than one memory component each working individually or with one another. Example types of memory may include random access memory (RAM), EEPROM, FLASH, suitable volatile storage devices, suitable non-volatile storage devices, persistent memory (e.g., read only memory (ROM), hard drive, Flash memory, optical disc memory, and/or other suitable persistent memory) and/or other suitable types of memory. The memory 38 may be or may include a non-transitory computer readable medium.

The I/O units 40 of the console 28 may include a single I/O component or more than one I/O component each working individually or with one another. Example I/O units 40 may be any type of communication port configured to communicate with other components of the circulatory system 10. Example types of I/O units 45 may include wired ports, wireless ports, radio frequency (RF) ports, Low-Energy Bluetooth ports, Bluetooth ports, Near-Field Communication (NFC) ports, HDMI ports, Wi-Fi ports, Ethernet ports, VGA ports, serial ports, parallel ports, component video ports, S-video ports, composite audio/video ports, DVI ports, USB ports, optical ports, and/or other suitable ports.

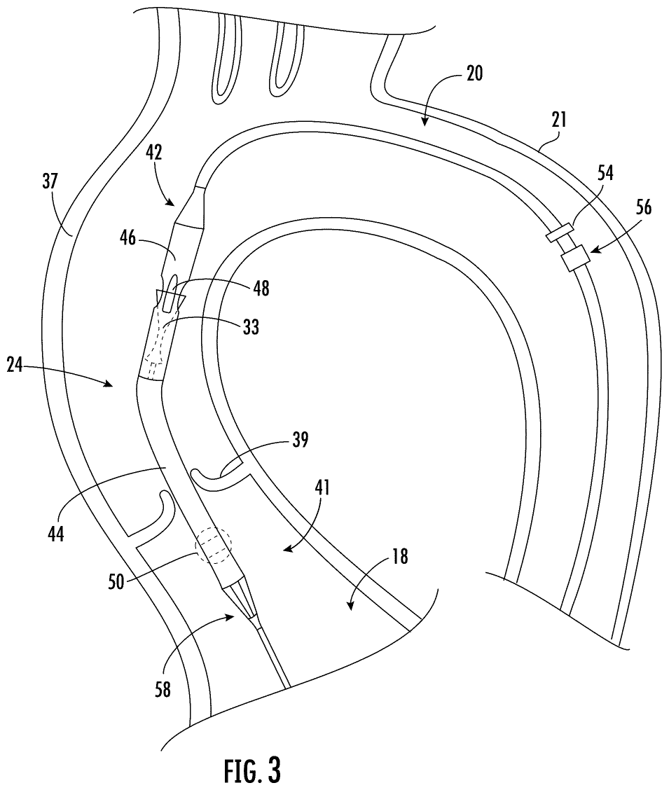

FIG. 3 illustrates the blood pump 24 of the percutaneous circulatory system 10 (shown in FIG. 1) extending from the ascending aorta 37 to the left ventricle 18 of a patient 16 (shown in FIG. 1). The blood pump 24 may include a cannula 44 having a proximal end attached to a distal end of an impeller housing 46. A proximal end 42 of the impeller housing 46 may be attached to a distal end of the catheter shaft 20. FIG. 3 illustrates that, in some examples, the blood pump 24 may be positioned within the heart 14 such that the cannula 44 passes through the aortic valve 39, whereby a distal end region 41 of the cannula 44 may be positioned within the left ventricle 18. As discussed herein, the blood pump 24 may be tracked over a guidewire to its position illustrated in FIG. 3.

FIG. 3 further illustrates that the circulatory support device 12 (shown in FIG. 1) may include one or more blood inlets 58 located on a distal end region 41 of the cannula 44, and one or more blood outlets 48 positioned along the impeller housing 46. In some examples, the blood pump 24 may be positioned within the heart 14 such that the one or more blood inlets 58 positioned along the distal end region 41 of the cannula 44 may be positioned in the left ventricle 18 and the one or more blood outlets 48 located along the impeller housing 46 may be positioned in the ascending aorta 37.

Additionally, the blood pump 24 may include an electrically powered motor that drives rotation of the impeller 33 which may be positioned within the impeller housing 46. In some examples, the motor may power the rotation of the impeller 33 via electromagnetic induction. The spinning impeller 33 may draw blood from the left ventricle 18 (via the one or more blood inlets 58 located on a distal region of the cannula 44) into the ascending aorta 37 (via the one or more blood outlets 48 located along the impeller housing 46). In other words, an electrically powered motor drives the impeller 33 to pump blood from the left ventricle 18 through the aortic valve 39 and into the ascending aorta 37.

Additionally, the circulatory support device 12 may include one or more transducers, sensors, etc. coupled to the cannula 44 and/or the catheter shaft 20. The one or more transducers coupled to the cannula 44 and/or the catheter shaft 20 may be designed to directly sense (e.g., measure, determine, calculate, generate, etc.) various therapeutic parameters (e.g., blood velocity, vessel diameter, etc.), whereby the direct measurement of the various therapeutic parameters may also be utilized to indirectly determine (e.g., calculate) the position of the circulatory support device within the blood vessel and/or other cardiac parameters (e.g., blood volumetric flow rate, cardiac output, ventricular ejection fraction, etc.).

FIG. 3 illustrates that, in some examples, the percutaneous circulatory system 10 may include an imaging transducer 50 (e.g., ultrasound transducer, ultrasound sensor, etc.) positioned along a distal end region of the cannula 44. For example, the imaging ultrasound sensor 50 may be positioned along the outside of the wall of the cannula 44 proximal to the blood inlets 58. As will be discussed in greater detail below, the imaging ultrasound sensor 50 may be designed to directly measure the diameter, volume, etc. of a body vessel (e.g., ascending aorta 37) and/or body structure (e.g., left ventricle 18, the aortic valve 39, etc.) within which the cannula 44 may be positioned. The ultrasound sensor 50 may also be used to identify ventricular structures during device placement, dwell, or re-positioning to determine appropriate device positioning for optimum therapy delivery.

FIG. 3 further illustrates that, in some examples, the percutaneous circulatory system 10 may also include a velocity ultrasound transducer 54 (e.g., blood velocity transducer, blood velocity sensor, etc.) positioned along the catheter shaft 20. The velocity ultrasound transducer 54 may be configured to directly sense (e.g., measure, determine, calculate, etc.) the velocity of blood flowing within a blood vessel (e.g., aorta 21) within which the velocity ultrasound transducer 54 is positioned. As will be described in greater detail below, the velocity ultrasound transducer 54 may include a doppler ultrasound sensor mounted on the catheter 20. In other examples, the velocity ultrasound transducer 54 may include an optical flow sensor mounted on the catheter 20. In some examples, the velocity ultrasound transducer 54 may be positioned about 60 millimeters (mm) (2.36 inches) or more from the blood outlets 48 in order for the velocity ultrasound transducer 54 to operate in a more laminar flow regime. The velocity ultrasound transducer 54 may be designed to directly sense (e.g., measure, determine, calculate, etc.) the velocity of blood adjacent to the velocity ultrasound transducer 54. For example, the velocity ultrasound transducer 54 may be designed to directly measure the velocity of blood passing through the aorta 21.

FIG. 3 further illustrates that, in some examples, the percutaneous circulatory system 10 may include a vessel sizing ultrasound transducer 56 positioned along the catheter shaft 20. In some examples, the vessel sizing ultrasound transducer 56 may be positioned adjacent to the velocity ultrasound transducer 54. The vessel sizing ultrasound transducer 56 may be designed to sense (e.g., measure, determine, calculate, etc.) the diameter of body vessel (e.g., aorta 21) within which the vessel sizing ultrasound transducer 56 is positioned. FIG. 3 illustrates that the velocity ultrasound transducer 54 is positioned along the catheter shaft 20 such that it is closer to the blood pump 24 relative to the vessel sizing ultrasound transducer 56 (i.e., the velocity ultrasound transducer 54 is positioned distal of the vessel sizing ultrasound transducer 56 along the catheter shaft 20). In this configuration, the velocity ultrasound transducer 54 may transmit an ultrasound pulse which is directed into the flow of blood flowing toward the velocity ultrasound transducer 54 (i.e., transmitting an ultrasound pulse in a retrograde direction relative to blood flow). However, in other examples, the velocity ultrasound transducer 54 may be positioned along the catheter shaft 20 such that it is father away from the blood pump 24 relative to the vessel sizing ultrasound transducer 56 (i.e., the velocity ultrasound transducer 54 is positioned proximal of the vessel sizing ultrasound transducer 56 along the catheter shaft 20). When positioned farther away from the blood pump 24 relative to the vessel sizing ultrasound transducer 56, the velocity ultrasound transducer 54 may transmit an ultrasound pulse which is directed away from the blood pump 24 and along the flow of blood flowing away from the velocity ultrasound transducer 54 (i.e., transmitting an ultrasound pulse in an antegrade direction relative to blood flow).

Additionally, it can be appreciated that, in some examples, each of the velocity ultrasound transducer 54 and the vessel sizing ultrasound transducer 56 may be attached to an outer surface of the catheter shaft 20. In other examples, however, at least a portion of the velocity ultrasound transducer 54, the vessel sizing ultrasound transducer 56 or both the velocity ultrasound transducer 54 and the vessel sizing ultrasound transducer 56 may extend into the wall of the catheter shaft 20. For example, at least a portion of the thickness of the velocity ultrasound transducer 54, the vessel sizing ultrasound transducer 56 or both the velocity ultrasound transducer 54 and the vessel sizing ultrasound transducer 56 may extend into the wall of the catheter shaft 20 to a depth defined as a percentage of the overall wall thickness of the catheter shaft 20. Accordingly, at least a portion of the thickness of the velocity ultrasound transducer 54, the vessel sizing ultrasound transducer 56 or both the velocity ultrasound transducer 54 may extend into the wall of the catheter shaft 20 a distance that is about 5% to about 75% of the thickness of the wall of the catheter shaft 20, or about 10% to about 65% of the thickness of the wall of the catheter shaft 20, or about 15% to about 55% of the thickness of the wall of the catheter shaft 20, or about 20% to about 45% of the thickness of the wall of the catheter shaft 20, or about 25% to about 35% of the thickness of the wall of the catheter shaft 20, in some instances.

In some examples, the velocity ultrasound transducer 54, the vessel sizing ultrasound transducer 56 or both the velocity ultrasound transducer 54 and the vessel sizing ultrasound transducer 56 may be embedded (e.g., fully or partially embedded) in the wall of the catheter shaft 20. For example, the velocity ultrasound transducer 54, the vessel sizing ultrasound transducer 56 or both the velocity ultrasound transducer 54 and the vessel sizing ultrasound transducer 56 may be partially embedded in the wall of the catheter shaft 20 such that the velocity ultrasound transducer 54, the vessel sizing ultrasound transducer 56 or both the velocity ultrasound transducer 54 and the vessel sizing ultrasound transducer 56 extend into the wall of the catheter 20 such that an outer facing surface of the velocity ultrasound transducer 54, the vessel sizing ultrasound transducer 56 or both the velocity ultrasound transducer 54 and the vessel sizing ultrasound transducer 56 extend radially outward of the outer surface of the catheter shaft 20. In other instances, the velocity ultrasound transducer 54, the vessel sizing ultrasound transducer 56 or both the velocity ultrasound transducer 54 may be fully embedded in the wall of the catheter shaft 20 such that the velocity ultrasound transducer 54, the vessel sizing ultrasound transducer 56 or both the velocity ultrasound transducer 54 may extend into the wall of the catheter 20 such that an outer facing surface of the velocity ultrasound transducer 54, the vessel sizing ultrasound transducer 56 or both the velocity ultrasound transducer 54 and the vessel sizing ultrasound transducer 56 are flush with or recessed radially inward of the outer surface of the catheter shaft 20. Additionally, in some examples, the velocity ultrasound transducer 54, the vessel sizing ultrasound transducer 56 or both the velocity ultrasound transducer 54 and the vessel sizing ultrasound transducer 56 may include a membrane extending over (e.g., covering) the outer facing surface of the velocity ultrasound transducer 54, the vessel sizing ultrasound transducer 56 or both the velocity ultrasound transducer 54 and the vessel sizing ultrasound transducer 56, whereby the membrane extending over the outer facing surface of the velocity ultrasound transducer 54, the vessel sizing ultrasound transducer 56 or both the velocity ultrasound transducer 54 and the vessel sizing ultrasound transducer 56 may provide a smooth covering extending across the outer facing surface and the portions of the catheter shaft extending therefrom.

In other examples, the velocity ultrasound transducer 54, the sizing ultrasound transducer 56 or both the velocity ultrasound transducer 54 and the sizing ultrasound transducer 56 may be mounted on or within an acoustically transparent housing or other type of housing. In some examples, the housing may be attached to the catheter shaft 20 via an adhesive. In other examples, the housing may be attached to the catheter shaft 20 via a chemical bonding process. In other examples, the housing may be attached to the catheter shaft via a heat-shrink sleeve placed around the housing and the catheter shaft 20. In other examples, the housing may be attached to the catheter shaft via molding the housing onto the catheter shaft 20 and/or molding another component onto the catheter shaft 20 to secure the housing thereto. In other examples, the housing may be attached to the catheter shaft 20 via a re-flow process whereby the catheter shaft 20 may be heated and reflowed around a portion of the housing to secure the housing to the catheter shaft 20. These are only exemplary. Other means of mounting or attaching the housing to the catheter shaft 20 are also contemplated.

The housing may be configured to include materials having material properties that do not interfere with the ultrasound signals passing therethrough. For example, the housing may be formed from a material having an impedance that substantially matches the impedance of blood passing by the housing. Additionally, the housing may be configured to accommodate any type and/or orientation of an ultrasound signal transmitted by the velocity ultrasound transducer 54 and/or the sizing ultrasound transducer 56. The housing may be streamlined such that blood or other bodily fluids may flow freely around the velocity ultrasound transducer 54 and/or the sizing ultrasound transducer 56. Additionally, a streamlined housing may be configured to permit the velocity ultrasound transducer 54 and/or the sizing ultrasound transducer 56 to be easily inserted into an access sheath without the edges, etc. of velocity ultrasound transducer 54 and/or the sizing ultrasound transducer 56 getting caught up on internal components of the access sheath.

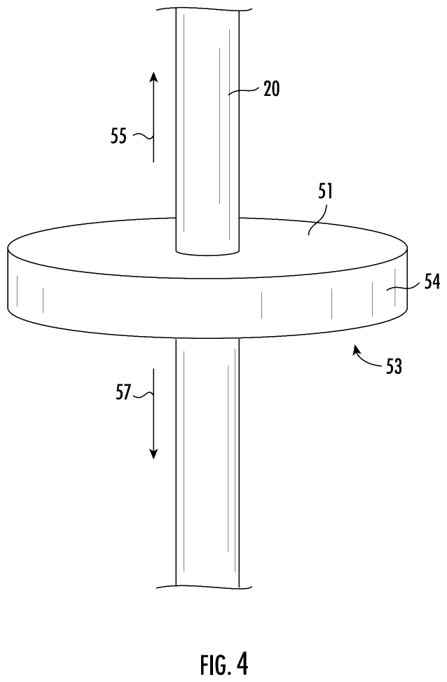

FIG. 4 illustrates the example velocity ultrasound transducer 54 shown FIG. 3. In some examples, the velocity ultrasound transducer 54 may operate as a doppler ultrasound transducer. Accordingly, the doppler ultrasound transducer may transmit (e.g., emit) ultrasound pulses (e.g., sound waves) when positioned within a body vessel (e.g., aorta 21). The emitted ultrasound pulses may be scattered and/or reflected by the flowing blood, thereby causing a frequency shift between the transmitted ultrasound pulses and the reflected ultrasound pulses. The change in frequency of the reflected ultrasound pulses received by the ultrasound transducer 54 (any of the ultrasound transducers described may be operated as both a transmitter and receiver) may be proportional to the velocity of the blood flowing through the body vessel and may provide an accurate measure for the velocity of the blood within the body vessel (e.g., the aorta 21).

As discussed herein, in other examples, the velocity sensor 54 may alternatively be a fiber optic flow sensor. Accordingly, a fiber optic flow sensor may include a laser which delivers pulsed heat injection a known distance adjacent to the temperature sensing portion of the fiber optic flow sensor. The injected heat may elevate the temperature of blood adjacent to the velocity ultrasound transducer 54 and create a temperature distribution of the blood flowing within the body vessel. Accordingly, sensing the temperature of blood a known distance downstream from the laser pulse with a known power may enable an accurate estimate of the velocity of blood with the body vessel.

FIG. 4 illustrates the velocity ultrasound transducer 54 may be configured to transmit ultrasound pulses from the surface 51. In this configuration, the surface 51 may be defined as an ultrasound transmission face 51 whereby ultrasound pulses are transmitted away from the surface 51 in a distal direction. The transmission face 51 may be substantially perpendicular to the longitudinal axis of the catheter shaft 20 to which the blood velocity ultrasound transducer 54 is attached. Referring to FIG. 3, it can be appreciated that the transmission face 51 may face toward the blood pump 24, and therefore, blood flowing through the aorta 21 may flow toward the transmission face 51. Accordingly, it can be appreciated that ultrasound transducer 54 may be mounted on the shaft 20 such that an ultrasound signal (depicted by the arrow 55) transmitted by the transmission face 51 may be directed into the flow of blood passing within the aorta 21 (i.e., transmitting an ultrasound pulse in a retrograde direction relative to blood flow).

In some examples, the velocity ultrasound transducer 54 may include a backing layer and/or backing material which is configured to enhance the acoustic performance of the velocity ultrasound transducer 54. For example, in a configuration in which the velocity ultrasound transducer 54 is positioned closer to the blood pump 24 relative to the sizing ultrasound transducer 56 (such as the configuration shown in FIG. 3), the velocity ultrasound transducer 54 may include a backing material positioned along the surface 53. It can be appreciated that the backing material may absorb an ultrasound wave or ultrasound echo propagating away from a piezoelectric element positioned within the velocity ultrasound transducer 54. If the ultrasound pulse wave is reflected back toward the piezoelectric element, it can cause noise in the ultrasound data. A backing layer may prevent backward emitted sound waves to echo and ring back into the velocity ultrasound transducer 54 for detection.

In other examples, such as a configuration in which the velocity ultrasound transducer 54 is positioned farther away from the blood pump 24 relative to the sizing ultrasound transducer 56 (e.g., a configuration in which the positions of the velocity ultrasound transducer 54 and the sizing ultrasound transducer 56 are switched in FIG. 3), the surface 53 may be defined as an ultrasound transmission face 53 whereby ultrasound pulses are transmitted away from the surface 53 in a proximal direction. The transmission 53 face may be substantially perpendicular to the longitudinal axis of the catheter shaft 20 to which the blood velocity ultrasound transducer 54 is attached. In this configuration, it can be appreciated that the ultrasound transmission face 53 may face away from the blood pump 24, and therefore, blood flowing through the aorta 21 may flow away from the transmission face 53. It can further be appreciated that an ultrasound signal (depicted by the arrow 57) transmitted by the transmission face 53 may be directed away from the blood pump 24 and with the flow of blood passing within the aorta 21 (i.e., transmitting an ultrasound pulse in an antegrade direction relative to blood flow). Like that described above, it can be appreciated that a velocity ultrasound transducer 54 which transmits ultrasound pulses from a transmission face 53 may include a backing layer positioned along the face 51. The backing layer may prevent backward emitted sound waves to echo and ring back into the velocity ultrasound transducer 54 for detection.

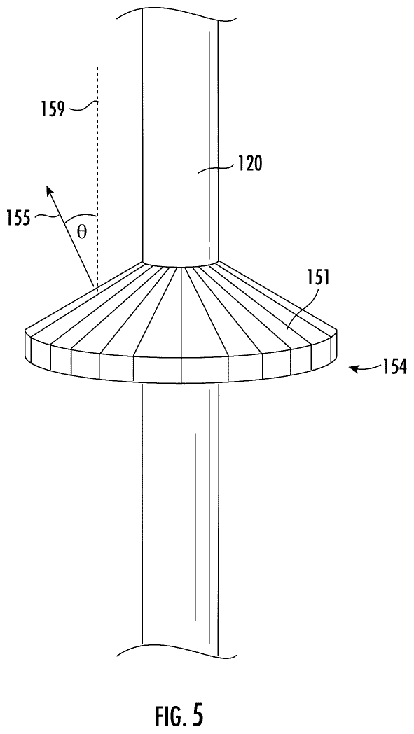

FIG. 5 illustrates another example velocity ultrasound transducer 154 attached to a catheter shaft 120. The ultrasound transducer 154 and the catheter shaft 120 may be similar in form and function to the velocity ultrasound transducer 54 and catheter shaft 20 described herein. However, FIG. 5 further illustrates the ultrasound transducer 154 may include a transmission face 151 which may face toward the blood pump 24 away from the vessel sizing transducer 56, and therefore, blood flowing through the aorta 21 may flow toward the first transmission face 151, and the vessel sizing transducer 56 would not interfere with the measurements. Further, FIG. 5 illustrates that the transmission face 151 may be angled relative to a longitudinal axis 159 which is parallel to the catheter shaft 120. It can be appreciated that an ultrasound pulse (depicted by the arrow 155) transmitted from the first transmission face 151 may propagate at an angle θ relative to the longitudinal axis 159. In some examples, the angle θ may be about 0 to about 60 degrees, or about 5 to about 50 degrees, or about 10 to about 40 degrees, or about 15 to about 35 degrees, or about 20 to about 30 degrees, or approximately 15 degrees. Transmitting the ultrasound pulse at an angle θ relative to the longitudinal axis 159 may prevent interference from the catheter shaft 120 during both the transmission and reception of the ultrasound signal from the first transmission face 151. Further, it can be appreciated that blood velocity values derived from pulses transmitted from the first transmission face 151 may be divided by the cosine of the angle θ to determine the blood velocity value which is parallel to the catheter shaft 120.

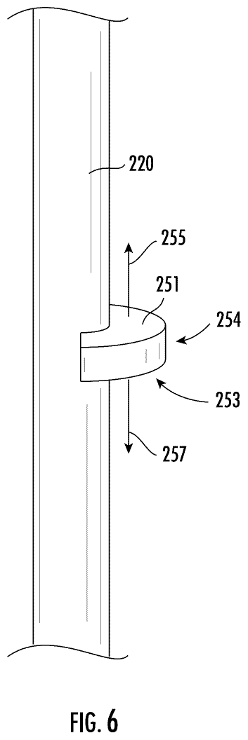

FIG. 6 illustrates another example velocity ultrasound transducer 254 attached to a catheter shaft 220. The ultrasound transducer 254 and the catheter shaft 220 may be similar in form and function to the ultrasound transducer 54 and catheter shaft 20 described herein. However, the velocity ultrasound transducer 254 may have a cubic or rectangular block-like shape and be mounted on the side of the catheter, or as illustrated in FIG. 6, extend circumferentially around only a portion of the catheter shaft 220. The velocity ultrasound transducer 254 may include a doppler ultrasound sensor and/or an optical flow sensor mounted on the catheter shaft 220.

FIG. 6 illustrates the velocity ultrasound transducer 254 may be configured to transmit ultrasound pulses from the surface 251. In this configuration, the surface 251 may be defined as an ultrasound transmission face 251 whereby ultrasound pulses are transmitted away from the surface 251 in a distal direction. The transmission face 251 may be substantially perpendicular to the longitudinal axis of the catheter shaft 220 to which the blood velocity ultrasound transducer 254 is attached. Referring to FIG. 3, it can be appreciated that the transmission face 251 may face toward the blood pump 24, and therefore, blood flowing through the aorta 21 may flow toward the transmission face 251. Accordingly, it can be appreciated that ultrasound transducer 254 may be mounted on the shaft 220 such that an ultrasound signal (depicted by the arrow 255) transmitted by the first transmission face 251 may be directed into the flow of blood passing within the aorta 21 (i.e., transmitting an ultrasound pulse in a retrograde direction relative to blood flow).

In some examples, the velocity ultrasound transducer 254 may include a backing layer and/or backing material which is configured to enhance the acoustic performance of the velocity ultrasound transducer 254. For example, in a configuration in which the velocity ultrasound transducer 254 is positioned closer to the blood pump 24 relative to the sizing ultrasound transducer 56 (such as the configuration shown in FIG. 3), the velocity ultrasound transducer 254 may include a backing material positioned along the surface 253. It can be appreciated that the backing material may absorb an ultrasound wave or ultrasound echo propagating away from a piezoelectric element positioned within the velocity ultrasound transducer 254. If the ultrasound pulse wave is reflected back toward the piezoelectric element, it can cause noise in the ultrasound data. A backing layer may prevent backward emitted sound waves to echo and ring back into the velocity ultrasound transducer 254 for detection.

In other examples, such as a configuration in which the velocity ultrasound transducer 254 is positioned farther away from the blood pump 24 relative to the sizing ultrasound transducer 56 (e.g., a configuration in which the positions of the velocity ultrasound transducer 254 and the sizing ultrasound transducer 56 are switched in FIG. 3), the surface 253 may be defined as an ultrasound transmission face 253 whereby ultrasound pulses are transmitted away from the surface 253 in a proximal direction. The transmission 253 face may be substantially perpendicular to the longitudinal axis of the catheter shaft 220 to which the blood velocity ultrasound transducer 254 is attached. In this configuration, it can be appreciated that the ultrasound transmission face 253 may face away from the blood pump 24, and therefore, blood flowing through the aorta 21 may flow away from the transmission face 253. It can further be appreciated that an ultrasound signal (depicted by the arrow 257) transmitted by the transmission face 253 may be directed away from the blood pump 24 and with the flow of blood passing within the aorta 21 (i.e., transmitting an ultrasound pulse in an antegrade direction relative to blood flow). Like that described above, it can be appreciated that a velocity ultrasound transducer 254 which transmits ultrasound pulses from a transmission face 253 may include a backing layer positioned along the face 251. The backing layer may prevent backward emitted sound waves to echo and ring back into the velocity ultrasound transducer 254 for detection.

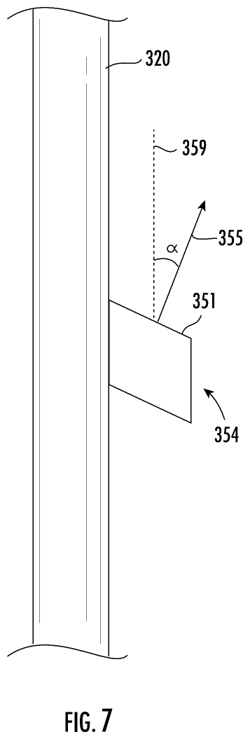

FIG. 7 illustrates another example velocity ultrasound transducer 354 attached to a catheter shaft 320. The ultrasound transducer 354 and the catheter shaft 320 may be similar in form and function to the velocity ultrasound transducer 54 and catheter shaft 20 described herein. However, as illustrated in FIG. 7, the velocity ultrasound transducer 354 may extend circumferentially around only a portion of the catheter shaft 320. The velocity ultrasound transducer 354 may include a doppler ultrasound sensor and/or an optical flow sensor mounted on the catheter shaft 320.

FIG. 7 further illustrates the velocity ultrasound transducer 354 of FIG. 6 may include a first transmission face 351 which may face toward the blood pump 24, and therefore, blood flowing through the aorta 21 may flow toward the first transmission face 351. Further, FIG. 7 illustrates that the first transmission face 351 may be angled relative to a longitudinal axis 359 which is parallel to the catheter shaft 320. It can be appreciated that an ultrasound pulse (depicted by the arrow 355) transmitted from the first transmission face 351 may propagate at an angle α relative to the longitudinal axis 359. In some examples, the angle θ may be about 0 to about 60 degrees, or about 5 to about 50 degrees, or about 10 to about 40 degrees, or about 15 to about 35 degrees, or about 20 to about 30 degrees, or approximately 15 degrees. Transmitting the ultrasound pulse at an angle α relative to the longitudinal axis 359 may prevent interference from the catheter shaft 320 during both the transmission and reception of the ultrasound signal from the first transmission face 351. Further, it can be appreciated that blood velocity values derived from pulses transmitted from the first transmission face 351 may be divided by the cosine of the angle α to determine the blood velocity value which is parallel to the catheter shaft 320.

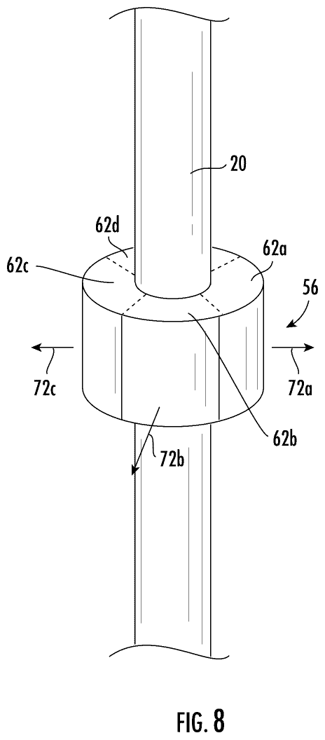

FIG. 8 illustrates the vessel sizing ultrasound transducer 56 shown in FIG. 3. The vessel sizing ultrasound transducer 56 may include an imaging array having one or more distinct ultrasound imaging transducer elements 62a-62d coupled to and extending circumferentially around the catheter shaft 20. For example, FIG. 8 illustrates that the ultrasound transducer 56 may include a first imaging transducer element 62a, a second imaging transducer element 62b, a third imaging transducer element 62c and a fourth imaging transducer element 62d. It can be appreciated that the vessel sizing ultrasound transducer may include 1, 2, 3, 4, 5, 6, 7, 8 or more imaging transducer elements.

It can be further appreciated that each of the imaging transducer elements 62a-62d may be configured to transmit an ultrasound signal which is substantially normal (e.g., perpendicular) to the lateral facing surface of the respective imaging transducer element 62a-62d. For example, FIG. 8 illustrates the ultrasound transducer element 62a transmitting an ultrasound signal 72a substantially perpendicular to a lateral facing surface of the transducer element 62a, the ultrasound transducer 62b element transmitting an ultrasound signal 72b substantially perpendicular to a lateral facing surface of the transducer element 62b and the ultrasound transducer element 62c transmitting an ultrasound signal 72c substantially perpendicular to a lateral facing surface of the transducer element 62c. Further, the ultrasound transducer element 62d may also transmit an ultrasound signal substantially perpendicular to its lateral facing surface, but is hidden from view in FIG. 8. Accordingly, the ultrasound transducer elements 62a-62d may sequentially transmit two or more ultrasound signals, whereby each of the ultrasound signals propagate radially outward and contact different portions of the body vessel (e.g., aorta) within which the imaging transducer elements 62a-62d are positioned.

It can be appreciated that transmitting multiple ultrasound signals from ultrasound transducer elements 62a-62d spaced at varying distancing from the body vessel wall may be utilized to determine diameter of the body vessel within which the ultrasound transducer elements 62a-62d are positioned. For example, the ultrasound transducer elements 62a-62d may both transmit ultrasound signals toward a vessel wall, as well as detect ultrasound signals reflected back to the ultrasound transducer elements 62a-62d after hitting the vessel wall. In general, ultrasound signals received by the ultrasound transducer elements 62a-62d, respectively, may generate electrical signals that are sent to a processor 36. Using the speed of sound and the time of each signal's return, the processor may calculate the distance from the ultrasound transducer elements 62a-62d to the vessel wall. These distances are then used to determine the diameter of the body vessel (e.g., aorta 21) within which the ultrasound transducer elements 62a-62d are positioned.

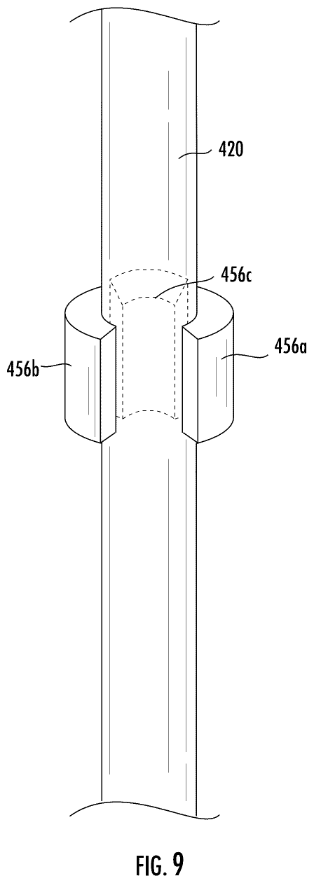

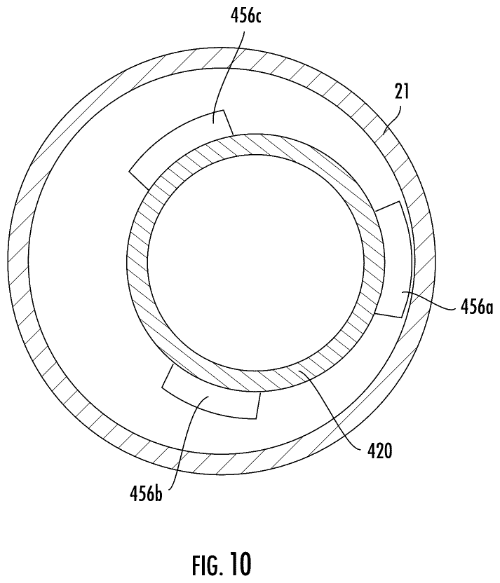

FIGS. 9-10 illustrates multiple vessel sizing ultrasound transducers 456a-456c coupled to the outer surface of a catheter shaft 420. The vessel sizing ultrasound transducers 456a-456c and the catheter shaft 420 may be similar in form and function to the vessel sizing ultrasound transducer 56 and catheter shaft 20 described herein. For example, FIG. 9 illustrates three ultrasound transducers 456a-456c positioned along the outer surface of the catheter shaft 420. It can be appreciated that the ultrasound transducers 456a-456c may be circumferentially spaced equidistant from one another around the outer surface of the catheter shaft 420. For example, the sensors 456a, 456b, 456c shown in FIGS. 9-10 may be circumferentially spaced substantially 120 degrees from one another. It can be further appreciated that the circulatory support device 10 (shown in FIG. 1) may include 1, 2, 3, 4, 5, 6, 7, 8, 9, 10 or more vessel sizing ultrasound transducers positioned adjacent one another at a given longitudinal location along the catheter shaft 20. At a given longitudinal location, the vessel sizing ultrasound transducers may be circumferentially spaced substantially equidistant from one another or they may not be spaced equidistant from one another. Further, one or more of any of the vessel sizing ultrasound transducers described herein may be either attached directly to an outer surface of a portion of the catheter shaft 20 or the one or more of any of the vessel sizing ultrasound transducers described herein may be embedded within the wall of the catheter shaft 20. Additionally, in some examples, the surfaces of the transducers 456a, 456b, 456c may be flat or curved.

FIG. 10 illustrates an end view of the ultrasound transducers 456a-456c positioned within the aorta 21. As described herein with respect to FIG. 8, FIG. 10 illustrates that multiple ultrasound signals may be transmitted from the ultrasound transducers 456a-456c which may be spaced at varying distancing from the body vessel wall. The ultrasound signals may be utilized to determine diameter of the body vessel within which the ultrasound transducers are positioned. For example, the ultrasound transducers 456a-456c may both transmit ultrasound signals toward the wall of the aorta 21, as well as detect ultrasound signals reflected back to the ultrasound transducers 456a-456c after hitting the vessel wall. In general, ultrasound signals received by the ultrasound transducers 456a-456c, respectively, may generate electrical signals that are sent to a processor 36. Using the speed of sound and the time of each signal's return, the processor may calculate the distance from the ultrasound transducer 456a-456c to the vessel wall. These distances are then used to determine the diameter of the body vessel (e.g., aorta 21) within which the ultrasound transducers 456a-456c are positioned.

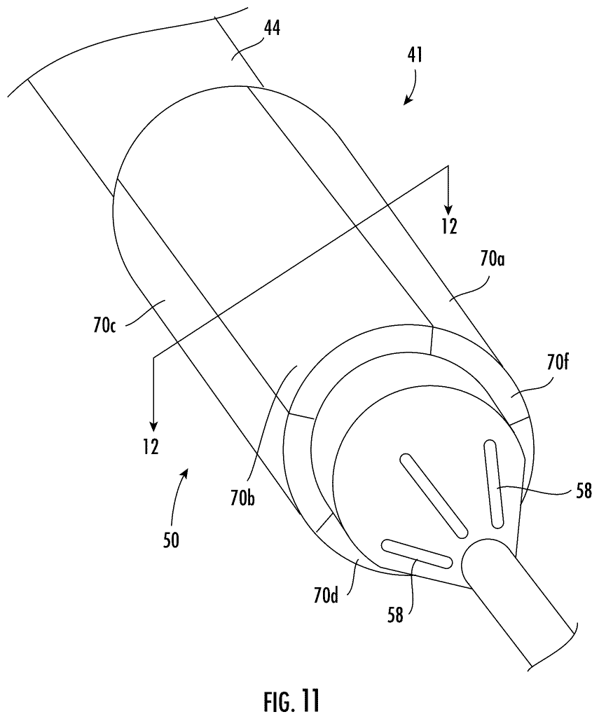

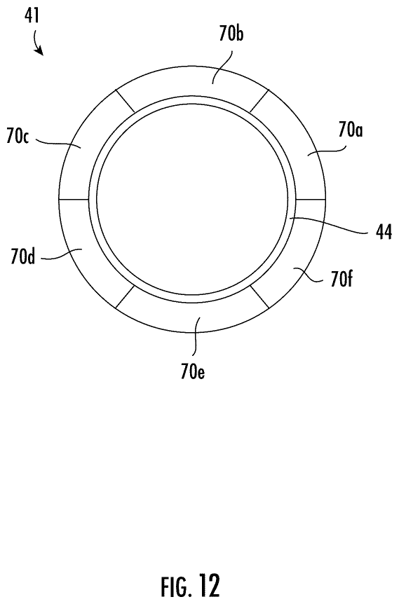

FIG. 11 illustrates the distal end region 41 of the cannula 44 including the imaging ultrasound transducer 50 positioned on an outside surface of the cannula 44. In other words, in some examples, the imaging ultrasound transducer 50 may be positioned such that it contacts and extends circumferentially along an outer surface of the wall of the cannula 44. In yet other examples, the imaging ultrasound transducer 50 may be spaced away from an outer surface of the cannula 44. Additionally, FIG. 12 illustrates that the imaging ultrasound transducer 50 may be positioned proximal to the one or more blood inlets 58 positioned along the distal end region 41 of the cannula 44.

FIGS. 11-12 illustrate that the imaging ultrasound transducer 50 may include an imaging array having one or more ultrasound imaging transducer elements 70a-70f coupled to and extending circumferentially around the cannula 44. FIG. 12 illustrates a cross-sectional view taken along line 12-12 of FIG. 11. FIG. 12 illustrates the ultrasound imaging transducer elements 70a-70f coupled to and extending circumferentially around the cannula 44. For example, FIG. 11 illustrates that that the imagining ultrasound transducer 50 may include a first imaging transducer element 70a, a second imagining transducer element 70b, a third imaging transducer element 70c, a fourth imaging transducer element 70d, a fifth imaging transducer element 70e and a sixth imaging transducer element 70f extending circumferentially around the cannula 44. It can be appreciated that the imaging ultrasound transducer 50 may include 1, 2, 3, 4, 5, 6, 7, 8, 9, 10, 20, 30, 40, 50, 80, 100 or more imaging transducer elements. In some examples, it can be appreciated that an imaging transducer may be positioned along the conical portion (e.g., angled portion) of the cannula 44 adjacent to the blood inlets 58. A transducer positioned along the conical portion of the cannula 44 may transmit an ultrasound signal (or a portion of an ultrasound signal) in a distal direction relative to the imaging ultrasound transducer 50. It can be further appreciated that each of the imaging transducer elements 70a-70f may be configured to transmit an ultrasound signal which is substantially normal (e.g., perpendicular) to the lateral facing surface of the respective imaging transducer imaging transducer elements 70a-70f. Accordingly, the ultrasound transducer elements 70a-70f may simultaneously or sequentially transmit two or more ultrasound signals, whereby each of the ultrasound signals propagate radially outward and contact different portions of the body vessel (e.g., left ventricle 18, ascending aorta 37) and surrounding tissues within which the imaging transducer elements 70a-70f are positioned.

It can be appreciated that transmitting multiple ultrasound signals from ultrasound transducer elements 70a-70f spaced at varying distancing from the body vessel wall may be utilized to image the tissues within which the ultrasound transducer elements 70a-70f are positioned. For example, the ultrasound transducer elements 70a-70f may both transmit ultrasound signals, as well as detect ultrasound signals reflected back to the ultrasound transducer elements 70a-70f after various tissues. A portion of the ultrasound signal may be reflected, and a portion may be transmitted to allow reflection off deeper anatomy. In general, ultrasound signals received by the ultrasound transducer elements 70a-70f, respectively, may generate electrical signals that are sent to a processor 36. Using the speed of sound and the time of each signal's return, the processor may calculate the distance from the ultrasound transducer elements 70a-70f to various tissues and anatomical structures. These distances are then used to produce an image of the vessel and surrounding tissues within which the ultrasound transducer elements 70a-70f are positioned.

Additionally, it can be appreciated that the processing components 36 of the percutaneous circulatory system 10 may include an algorithm capable of receiving data from the ultrasound system corresponding to the diameter of a body vessel (e.g., the ascending aorta, descending aorta, etc.) adjacent to a flow sensor (e.g., the flow sensor 50 and the flow sensor 52). Further, the data received from the ultrasound system may be utilized by the processing components 36 of the percutaneous circulatory system 10 to calculate the cardiac output of the heart 14.

It can be appreciated that any of the ultrasound transducers and/or sensors described herein may send signals to the console 28 and/or the processing components 36 via a wireless connection (e.g., a Bluetooth connection). In other examples, any of the ultrasound transducers and/or sensors described herein may be hardwired to the console 28 and/or the processing components 36.

Additionally, the ultrasound transducers 54, 56 described herein may be sized and shaped to permit the blood to flow across the surface area thereof without substantially impeding or disturbing the velocity and/or fluid dynamics of the blood flow. It can be appreciated that the ultrasound transducers 54, 56 described herein may include any shape, including a circular, ovular, square, triangular, polygonal, star-shaped, or any combinations thereof. Additionally, the ultrasound transducers 54, 56 described herein may be covered by an acoustically transparent material. Further, the acoustically transparent material covering the transducers may be tapered to reduce the impact on the flow of blood. Further, any of the ultrasound transducers 50, 54, 56 described herein may be formed from a variety of different materials including, but not limited to, a PZT (lead zirconate titanate) ceramic, a single crystal, a piezo-polymer composite or piezoelectric.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A cardiac pump system, comprising:
a catheter shaft having a distal end region coupled to a cardiac pump, wherein the cardiac pump includes an impeller housing, a cannula and an impeller, wherein the cannula includes a distal end region and a proximal end region, wherein the distal end region of the cannula is configured to be positioned in a left ventricle of a heart;
a first ultrasound transducer coupled to the catheter shaft, wherein the first ultrasound transducer is configured to directly measure a velocity of blood flowing adjacent to the first ultrasound transducer; and
a second ultrasound transducer coupled to the catheter shaft, wherein the second ultrasound transducer is configured to transmit a first ultrasound signal toward a wall of a body vessel and receive a reflected portion of the first ultrasound signal reflected from the wall of the body vessel.

2. The cardiac pump system of claim 1, further comprising a console coupled to the catheter shaft, wherein the console includes a processor, and wherein the console is configured to receive the reflected portion of the first ultrasound signal from the second ultrasound transducer.

3. The cardiac pump system of claim 2, wherein the processor is configured to utilize the reflected portion of the first ultrasound signal to determine a diameter of the body vessel adjacent to the second ultrasound transducer.

4. The cardiac pump system of claim 3, wherein the processor is configured to calculate a flow rate of blood passing through the body vessel based on the velocity of blood measured by the first ultrasound transducer and the diameter of the body vessel determined by the processor.

5. The cardiac pump system of claim 1, wherein the first ultrasound transducer is positioned adjacent to the second ultrasound sensor.

6. The cardiac pump system of claim 1, wherein the first ultrasound transducer and the second ultrasound transducer are attached to an outer surface of the catheter shaft.

7. The cardiac pump system of claim 1, wherein the first ultrasound transducer and the second ultrasound transducer are attached to a housing attached to the catheter shaft.

8. The cardiac pump system of claim 1, wherein the first ultrasound transducer, the second ultrasound transducer or both the first ultrasound transducer and the second ultrasound transducer extend at least partially into a wall of the catheter shaft.

9. The cardiac pump system of claim 1, wherein the first ultrasound transducer is configured to utilize doppler ultrasound to directly measure the velocity of blood flowing adjacent to the first ultrasound transducer.

10. The cardiac pump system of claim 1, wherein one or more optical fibers are utilized to directly measure the velocity of blood flowing adjacent to the first ultrasound transducer.

11. The cardiac pump system of claim 2, wherein the second ultrasound sensor is configured to transmit a second ultrasound signal toward the body vessel wall and receive a reflected portion of the second ultrasound signal reflected from the vessel wall, and wherein the console is configured to receive the reflected portion of the second ultrasound signal from the second ultrasound transducer, and wherein the processor is configured to utilize the reflected portion of the first ultrasound signal and the reflected portion of the second ultrasound signal to determine a diameter of the body vessel adjacent the second ultrasound transducer.

12. The cardiac pump system of claim 1, wherein the first ultrasound transducer includes a first transmission face configured to transmit a velocity ultrasound signal, and wherein the first transmission face tapers away from the catheter shaft such that the velocity ultrasound signal transmitted by the first transmission face propagates away from the catheter shaft at an angle relative to a longitudinal axis of the catheter shaft.

13. The cardiac pump system of claim 12, wherein the first ultrasound transducer extends circumferentially around the catheter shaft.

14. The cardiac pump system of claim 1, wherein the second ultrasound transducer includes an array of individual ultrasound transducer elements.

15. A cardiac pump system, comprising:
a catheter shaft having a distal end region coupled to a cardiac pump, wherein the cardiac pump includes an impeller housing, a cannula and an impeller, wherein the cannula includes a distal end region and a proximal end region, wherein the distal end region of the cannula is configured to be positioned in a left ventricle of a heart;
a first ultrasound transducer coupled to the catheter shaft;
a second ultrasound transducer coupled to the catheter shaft, wherein the first ultrasound transducer is positioned adjacent to the second ultrasound sensor; and
a third ultrasound transducer positioned on an outer surface of the cannula.

16. The cardiac pump of claim 15, further comprising a fourth ultrasound transducer positioned on the outer surface of the cannula, and wherein the first ultrasound transducer and the second ultrasound sensor are coupled to a housing positioned on the catheter shaft.

17. A cardiac pump system, comprising:
a catheter shaft having a distal end region coupled to a cardiac pump, wherein the cardiac pump includes an impeller housing, a cannula and an impeller, wherein the cannula includes a distal end region and a proximal end region, wherein the distal end region of the cannula is configured to be positioned in a left ventricle of a heart;
a first ultrasound transducer coupled to the catheter shaft, wherein the first ultrasound transducer is configured to directly measure a velocity of blood flowing adjacent to the first ultrasound transducer;
a second ultrasound transducer coupled to the catheter shaft, wherein the second ultrasound transducer assembly is configured to transmit an ultrasound signal toward a wall of a body vessel and receive a reflected portion of the ultrasound signal reflected from the wall of the body vessel; and
a third ultrasound transducer positioned on an outer surface of the cannula.

18. The cardiac pump system of claim 17, wherein the third ultrasound transducer is configured to transmit an imaging ultrasound signal and receive a reflected portion of the imaging ultrasound signal reflected from blood or a body tissue.

19. The cardiac pump system of claim 18, further comprising a fourth ultrasound transducer positioned on the outer surface of the cannula, and wherein the fourth ultrasound transducer is circumferentially spaced 180 degrees from the third ultrasound transducer.

20. The cardiac pump system of claim 19, further comprising a console coupled to the catheter shaft, wherein the console includes a processor, wherein the processor is configured to generate a three-dimensional image based on the reflected portion of the imaging ultrasound signal received from the third ultrasound transducer.
